(19) United States
(12) Patent Application Publication
(10) Pub. No.: US 2006/0105986 A1
(43) Pub. Date: May 18, 2006

(54) Antimicrobial molecule

(75) Inventors: Richard Belanger, Quebec (CA); Caroline Labbe, Quebec (CA)

Correspondence Address: OGILVY RENAULT LLP, 1981 MCGILL COLLEGE AVENUE, SUITE 1600, MONTREAL, QC, H3A2Y3, CA

(73) Assignee: UNIVERSITE LAVAL, QUEBEC (CA)

(21) Appl. No.: 10/519,844

(22) PCT Filed: Jul. 16, 2003
(86) PCT No.: PCT/CA03/01080

§ 371 Date: Jul. 29, 2005

Related U.S. Application Data
(60) Provisional application No. 60/395,997, filed on Jul. 16, 2002.

(51) Int. Cl. A61K 31/7024; C12P 19/06; C07H 15/04

(52) U.S. Cl. 514/53; 536/116; 435/104

(57) ABSTRACT

The present invention relates to a compound having antimicrobial activities. The present invention also provides methods for the production and purification of flocculosin, the antimicrobial compound of the invention, including the steps of cultivating the microorganism Pseudozyma flocculosa. Analogs and derivatives of flocculosin are described herein.

### TECHNICAL FIELD

The present invention relates to antimicrobial compounds. The antimicrobial compounds can be used as medical, nutraceutical, and agricultural chemicals, for controlling growth of microorganisms.

### BACKGROUND ART

Organisms that cause infectious disease (bacteria, fungi, viruses, and other parasites) can contribute to and complicate many diseases. The worldwide use of antimicrobial agents to treat infectious diseases in humans, animals, plants as well as to control or treat other undesirable microorganisms has grown dramatically over the last forty to fifty years.

However, even considering the quantity of antimicrobial products available today, there is still a large place for new compound having antimicrobial activities. Furthermore, microbial resistance or tolerance to antimicrobial compounds, through the misuse or overuse of antimicrobials, has been a considerable problem in treating diseases. Moreover, existing antimicrobials can demonstrate unwanted toxicity in the treated patient, animal or plant. Therefore, the constant development of novel antimicrobials and uses thereof, such as combining new and existing antimicrobials, are necessary to treat and control infectious organisms as well as to eliminate or retard the onset of deleterious side effects such as microbial resistance or toxicity

To date, many antimicrobial substances have been isolated and characterized from bacteria and fungi and described as biological control agents. The diversity of these microbial products can be an invaluable source for the discovery of new agrochemicals and pharmaceuticals.

Pseudozyma flocculosa is a yeast-like fungus with biocontrol properties against powdery mildew fungi through antibiosis i.e. the release of antimicrobial compounds. This activity is attributable in part to the production of unusual extracellular fatty acids with antifungal properties. However, these fatty acids are highly unstable and will degrade rapidly after their release rendering them relatively inefficient when used alone.

Considering the state of the art for antimicrobial compounds, there is still an overall need to have new and effective means to treat or control infectious organisms through the development and use of novel antimicrobial compounds such as described herein bellow.

### DISCLOSURE OF INVENTION

One object of the present invention is to provide a compound having the formula (1): 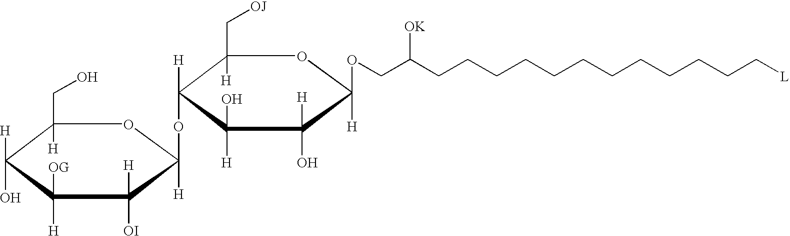
wherein G is H or 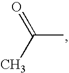
I is H or 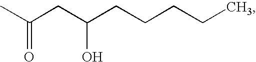
J is H or 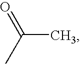
K represents H or 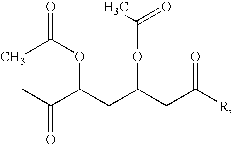
in which R is an hydroxyl (OH), an acyl, an alkyl, a methyl, an NH₂ group or a NH—R′ group, where R′ is an acyl or an alkyl; and L represents

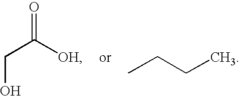

Another object of the invention is to provide compounds having the general formula (2): 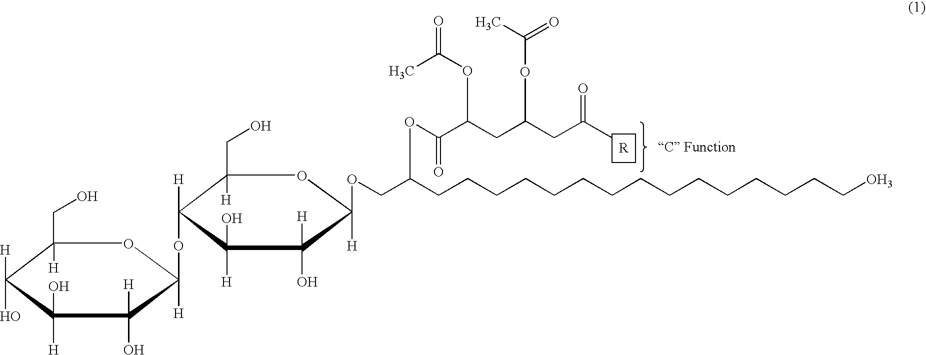
or an analog, a derivative or a salt thereof, wherein R can be an hydroxyl (OH), an acyl, an alkyl, a methyl, a NH₂ group or a NH—R′ group, where R′ is an acyl or an alkyl, the molecule being defined as flocculosin.

Yet another object of the invention relates to a compound having the general formula (3):

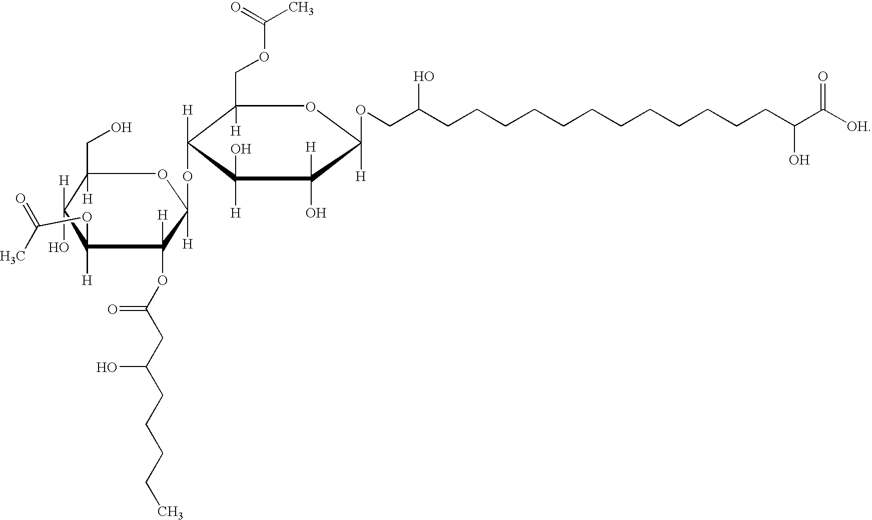

Another object of the present invention is to provide a compound having the previously described formulae, wherein R is an acyl or an alkyl such as methyl, which can be added to the flocculosin molecule by acylation, alkylation or methylation of the function defined as “C” function.

A further object of the present invention is to provide a compound having the previously described formulae, wherein R is a NH₂ group or a NH—R′ group, wherein R′ is an acyl or an alkyl, which can be added to the flocculosin molecule by amidation or amination of the function defined as “C” function.

Another aim of the present invention is to provide an analog, a derivative, or a salt of flocculosin.

Another object of the present invention is to provide an antimicrobial composition comprising flocculosin or an analog, a derivative, or a salt thereof, which can be found in association with another active compound or antimicrobial product.

For the purpose of the present invention the following terms are defined below.

The term “antimicrobial agent” as used herein is intended to mean any chemical or biologic agent that either destroys or inhibits the growth of microorganisms.

The term “antibiotic” as used herein is intended to mean a substance of microbial origin that has antimicrobial activity.

### BRIEF DESCRIPTION OF THE DRAWINGS

FIG. 1 illustrates the basic structure of flocculosin, where A is an acetyl, B is an acetyl, C is a carboxyl, D is an ester and E represent disaccharide hydroxyls.

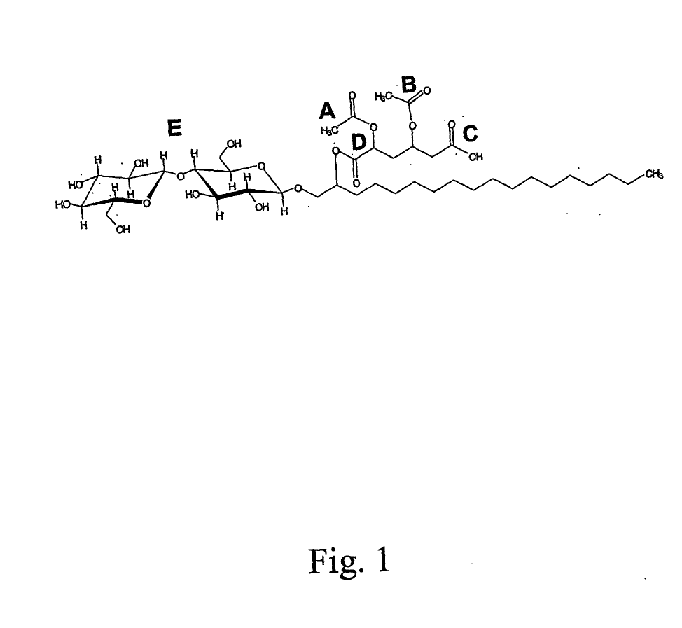

FIGS. 2A and 2B illustrate the protection of the cellobiose (disaccharide) hydroxyl groups using benzyl chloride (FIG. 2A) or methoxymethylether (FIG. 2B).

FIG. 3 illustrates the alkylation or arylation of the “C” function.

FIGS. 4A and 4B illustrate the esterification of the “C” function, which can be non-selective (FIG. 4A) or selective (FIG. 4B).

FIG. 5 illustrates the formation of an amide group on the “C” function.

FIG. 6 illustrates the methylation of the hydroxyl groups of the cellobiose.

FIG. 7 illustrates the efficacy of flocculosin against Candida albicans.

FIG. 8 illustrates the combined effect of flocculosin with Amphotericin B.

### MODES OF CARRYING OUT THE INVENTION

In accordance with the present invention, there is provided a new compound having antimicrobial activities. The compound, flocculosin, was isolated from Pseudozyma flocculosa.

As part of the ongoing investigation of Pseudozyma flocculosa, a novel and unusual molecule has been found to have antimicrobial activity and great stability, herein named flocculosin. The prior art references have not shown the existence of flocculosin and the use or any operable aspects of flocculosin.

According to one embodiment of the present invention, there is provided a compound represented by the general formula (1): 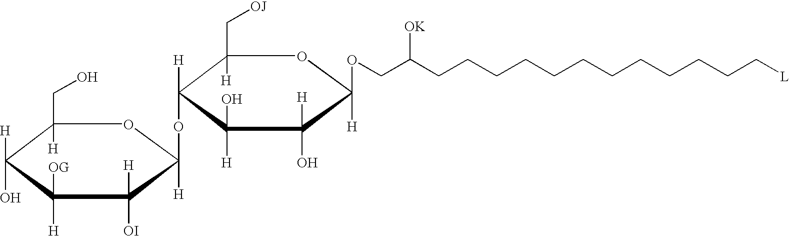
wherein G is H or 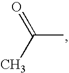
I is H or 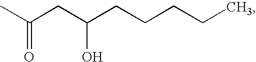
J is H or 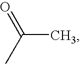
K represents H or 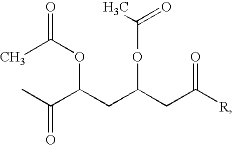
in which R is an hydroxyl (OH), an acyl, an alkyl, a methyl, an NH₂ group or a NH—R′ group, where R′ is an acyl or an alkyl; and L represents

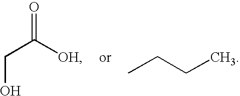

One embodiment of the present invention is to provide a compound having the general chemical structure of flocculosin as illustrated herein, wherein the R hydroxyl group (—OH) is replaced by an acyl, an alkyl or a methyl group, said group being added to the flocculosin molecule by arylation, alkylation or methylation of the function defined as “C” function.

Another embodiment of the present invention is to provide a compound having the general chemical structure of flocculosin, wherein the R hydroxyl group (—OH) is replaced by a NH₂ group or a NH—R′ group, wherein R′ is an acyl or an alkyl, the group being added to the flocculosin molecule by amidation or amination of the function defined as “C” function.

According to another embodiment of the present invention there are provided compounds derived from flocculosin that are modified according to methods exemplified herein.

Considering that flocculosin possesses antimicrobial activity against microorganisms pathogenic to plants and animals, they can be used for the treatment and prevention of infections caused by such organisms. Hosts treatable include plants and animals, particularly mammals and especially humans.

In animals and particularly humans, fungal infections may be cutaneous, subcutaneous, or systemic. Superficial mycoses include Tinea capitis, Tinea corporis, Tinea pedis, onychomycosis (e.g., nail fungus), perionychomycosis, pityriasis versicolor, oral thrush, and other candidoses such as vaginal, respiratory tract, biliary, eosophageal, and urinary tract candidoses. Systemic mycoses include systemic and mucocutaneous candidosis, cryptococcosis, aspergillosis, mucormycosis, paracoccidioidomycosis, blastomycosis, histoplasmosis, coccidioidomycosis, and sporotrichosis.

Examples of pathogenic organisms include, but are not limited to, dermatophytes (e.g., Microsporum canis, M. gypseum, M. distortum, M. audouinii, M. ferrugineum, M. rivalieri, M. filvum, M. cookei, M. vanbreuiseghemii, M. persicolor, Trichophyton rubrum, T. mentagrophytes, T. mengninii, T. nanum, T. schoenleinii, T. tonsurans, T. verrucosum, T. soudanense, T. violaceum, T. yaoundei, T. gourvilii, T. simii, T. ajelloi, Hendersonula toruloidea), yeasts (e.g., Candida albicans, C. tropicalis), Torulopsis glabrata, Epidermophyton floccosum, Malassezia furfur (Pityropsporon orbiculare, P. ovale), Cryptococcus neoformans, Aspergillus fumigatus and other Aspergillus spp., Zygomycetes (e.g., Rhizopus, Mucor), Paracoccidioides brasiliensis, Blastomyces dermatitidis, Histoplasma capuslatum, Coccidioides immitis, and Sporothrix schenckii.

The compounds according to the invention have a potent microbicidal activity and can be employed for controlling undesirable microorganisms, such as fungi and bacteria, in crop protection and in the protection of materials.

Fungicides are employed in crop protection for controlling Plasmodiophoromycetes, Oomycetes, Chytridiomycetes, Zygomycetes, Ascomycetes, Basidiomycetes and Deuteromycetes.

Bactericides are employed in crop protection for controlling Pseudomonadaceae, Rhizobiaceae, Enterobacteriaceae, Corynebacteriaceae and Streptomycetaceae.

Some pathogens causing fungal and bacterial diseases which come under the generic names listed above are mentioned as examples, but not by way of limitation:

Xanthomonas species, such as, for example, Xanthomonas campestris pv. oryzae;

Pseudomonas species, such as, for example, Pseudomonas syringae pv. lachrymans;

Erwinia species, such as, for example, Erwinia amylovora;

Pythium species, such as, for example, Pythium ultimum;

Phytophthora species, such as, for example, Phytophthora infestans;

Pseudoperonospora species, such as, for example, Pseudoperonospora humuli or Pseudoperonospora cubensis;

Plasmopara species, such as, for example, Plasmopara viticola;

Bremia species, such as, for example, Bremia lactucae;

Peronospora species, such as, for example, Peronospora pisi or P. brassicae;

Erysiphe species, such as, for example, Erysiphe graminis;

Sphaerotheca species, such as, for example, Sphaerotheca fuliginea;

Podosphaera species, such as, for example, Podosphaera leucotricha;

Venturia species, such as, for example, Venturia inaequalis;

Pyrenophora species, such as, for example, Pyrenophora teres or P. graminea (conidia form: Drechslera, syn: Helminthosporium);

Cochliobolus species, such as, for example, Cochliobolus sativus (conidia form: Drechslera, syn: Helminthosporium);

Uromyces species, such as, for example, Uromyces appendiculatus;

Puccinia species, such as, for example, Puccinia recondita;

Sclerotinia species, such as, for example, Sclerotinia sclerotiorum;

Tilletia species, such as, for example, Tilletia caries;

Ustilago species, such as, for example, Ustilago nuda or Ustilago avenae;

Pellicularia species, such as, for example, Pellicularia sasakii;

Pyricularia species, such as, for example, Pyricularia oryzae;

Fusarium species, such as, for example, Fusarium culmorum;

Botrytis species, such as, for example, Botrytis cinerea;

Septoria species, such as, for example, Septoria nodorum;

Leptosphaeria species, such as, for example, Leptosphaeria nodorum;

Cercospora species, such as, for example, Cercospora canescens;

Altemaria species, such as, for example, Altemaria brassicae; and

Pseudocercosporella species, such as, for example, Pseudocercosporella herpotrichoides.

The fact that the active compounds are well tolerated by plants at the concentrations required for controlling plant diseases permits the treatment of above-ground parts of plants, of propagation stock and seeds, and of the soil.

The active compounds according to the invention can be used with particularly good results for controlling diseases in viticulture and in fruit and vegetable growing, such as, for example against Venturia, Botrytis, Sclerotinia, Rhizoctonia, Uncinula, Sphaerotheca, Podosphaera, Altemaria and Colletotrichum species. Rice diseases, such as Pyricularia and Pellicularia species are likewise controlled with good results.

The active compounds according to the invention are also suitable for increasing the yield of crops. Moreover, they have reduced toxicity and are tolerated well by plants.

In the protection of materials, the compounds according to the invention can be employed for protecting industrial materials against infection with, and destruction by, undesired microorganisms.

Industrial materials in the present context are understood as meaning non-living materials which have been prepared for use in industry. For example, industrial materials which are intended to be protected by active compounds according to the invention from microbial change or destruction can be adhesives, sizes, paper and board, textiles, leather, wood, paints and plastic articles, cooling lubricants and other materials which can be infected with, or destroyed by, microorganisms. Parts of production plants, for example cooling-water circuits, which may be impaired by the proliferation of microorganisms may also be mentioned within the scope of the materials to be protected. Industrial materials which may be mentioned within the scope of the present invention are preferably adhesives, sizes, paper and board, leather, wood, paints, cooling lubricants and heat-transfer liquids, particularly preferably wood.

Microorganisms capable of degrading or changing the industrial materials which may be mentioned are, for example, bacteria, fungi, yeasts, algae and slime organisms. The active compounds or compositions according to the invention preferably act against fungi, in particular moulds, wood-discoloring and wood-destroying fungi (Basidiomycetes), and against slime organisms and algae.

Microorganisms of the following genera may be mentioned as examples: Alternaria, such as Alternaria tenuis, Aspergillus, such as Aspergillus niger, Chaetomium, such as Chaetomium globosum, Coniophora, such as Coniophora puetana, Lentinus, such as Lentinus tigrinus, Penicillium, such as Penicillium glaucum, Polyporus, such as Polyporus versicolor, Aureobasidium, such as Aureobasidium pullulans, Sclerophoma, such as Sclerophoma pityophila, Trichoderma, such as Trichoderma viride, Escherichia, such as Escherichia coli, Pseudomonas, such as Pseudomonas aeruginosa, and Staphylococcus, such as Staphylococcus aureus.

The dosage form and mode of administration as well as the dosage amount may be selected by the skilled artisan. Administration to a mammalian host may for example be oral, parenteral or topical. Administration to a plant host may be accomplished for example, by application to seed, foliage, other plant parts or to soil.

When flocculosin or the salts thereof are used as therapeutics, they can be administered alone or in a pharmaceutically suitable formulation containing in addition to the active ingredient one or more conventional carrier. Depending on the nature of the disease and/or route of administration, the composition of this invention can be formulated by known means.

Another embodiment of the present invention includes the use of flocculosin in combination with other active ingredients, including other antimicrobials. Flocculosin may be used in combination, in alternation or in a sequential way with active ingredients.

Examples of active ingredients which can be used in combination with flocculosin include, but are not limited to, allymines (e.g. amorolfine, butenafine, naftifine, terbinafine), azoles (e.g. fluconazole, itraconazole, ketoconazole, voriconazole, clotrimazole, econazole, miconazole, oxiconazole, sulconazole, terconazole, tioconazole), glucan synthesis inhibitors (e.g. caspofungin, other candins), polyenes (e.g. amphotericin B, nystatin, pimacin), griseofulvin, ciclopirox olamine, haloprogin, tolnaftate, undecylenate.

Depending on their particular physical and/or chemical properties, the active compounds can be converted to customary formulations, such as solutions, emulsions, suspensions, powders, foams, pastes, granules, aerosols and micro-encapsulations in polymeric substances and in coating compositions for seeds, and ULV cool and warm fogging formulations.

Example of pharmaceutical compositions include any solid (tablets, pills, capsules, granules, powder, etc.) or liquid (solutions, suspensions or emulsions) composition suitable for oral, topical or parenteral administration and they may contain the pure compound or a salt thereof or in combination with any carrier or other pharmaceutically active compounds. These compositions may need to be sterile when administered parenterally.

The dosage administered will depend upon the identity of the diseases, the type of host involved including its age, health and weight; the kind of concurrent treatment, if any; and the frequency of treatment and therapeutic ratio. Illustratively, dosage levels of the administered active ingredient are intravenous, 0.1 to ca. 200 mg/kg; intramuscular, 1 to about 500 mg/kg; orally, 5 to about 1000 mg/kg; and aerosol, 5 to about 1000 mg/kg of host body weight. Expressed in terms of concentration, an active ingredient can be present in the compositions of the present invention for localized use about the cutis, intranasally, pharyngolaryngeally, bronchially, intravaginally, rectally, or ocularly in a concentration of from about 0.01 to about 50%/w/w of the composition and preferably about 1 to about 20% w/w of the composition. Also, similarly, for parenteral use the invention can be used in a concentration of from about 0.05 to about 50% w/v of the composition and preferably from about 5 to about 20% w/v. Flocculosin and salts thereof used as active ingredients to be employed as antimicrobial agents for treatment of human and animal illness can be easily prepared in such unit dosage form with the employment of pharmaceutical materials which themselves are available in the art and can be prepared by established procedures. The appropriate solid or liquid vehicle or diluent may be selected and the composition prepared by methods known to the skilled artisan.

For agricultural applications, an antimicrobial compositions may be formed using the active ingredient as described herein in an inert carrier. If formulated as a solid, the ingredients may be mixed with such typical carriers as Fuller's earth, kaolin clays, silicas or other wettable iorganic diluents. Free-flowing dusts formulations may also be utilized by combining the dry active ingredients with finely divided solids such as talc, kieselguhr, pyrophyllite, clays, diatomaceous earth and the like.

The powders may also be applied as a suspension or solution, depending on the solubility in the liquid carrier. Pressurized sprays, typically aerosols with the active ingredient dispersed in a low-boiling dispersant solvent carrier may be used. Percentages of weight may vary according to the manner in which the composition is to be applied and formulation used. In general, the active ingredient will comprise 0.005% to 95% of the active ingredient by weight in the antimicrobial composition. The antibiotic composition may be applied with other ingredients including growth regulators, insecticides, herbicides, fertilizers and the like. Formulation of the active ingredients to assist applicability, ease, handling, maintain chemical stability and increase effectiveness may require addition of various materials. Solvents may be chosen on the basis of affecting the solubility of the active ingredient, fire hazard, and flash point, emulsifiability, specific gravity and economic considerations.

According to another embodiment of the present invention, any adjuvant may be added to enhance the active ingredients and can include surfactants which are anionic, cationic or nonionic. Stabilizers and antifreeze compounds will prolong storage. Additionally, synergists, stickers, spreaders and deodorant compounds can be added to improve the handling characteristics of the commercial formulation. Alternatively, the active ingredient can be combined with an inert carrier, such as calcium carbonate, and formed into a pill or other consumable delivery device, including controlled release devices intended to deliver metered doses of the active ingredient.

The inventive compound of the present invention may be employed also as antimicrobial agents useful in inhibiting the growth of microorganisms present or eradicating microorganisms on a surface or in a medium outside a living host. The inventive compound and/or its salts thereof may be employed, for example, as disinfectants for a variety of solid and liquid media susceptible to microbial growth. Suitable amounts of the inventive compound may be determined by methods known to the skilled artisan.

### EXAMPLES

The present invention will be more readily understood by referring to the following examples which are given to illustrate the invention rather than to limit its scope.

### EXAMPLE I

### Isolation and Identification of Flocculosin

Materials and Methods

Isolation and Purification

P. flocculosa wild-type (WT) strain PF-1 (ATCC 64874) was grown at 25° C. in petri dishes containing 20 ml of Czapek Dox broth (Difco, Sparks, Md.) supplemented with 0.4% Phytagel™ (Sigma, Steinheim, Germany) for 8 days. Cultures as well as medium were collected, freeze-dried, ground to a fine powder, and subjected to extraction in 80% methanol (MeOH) (ca. 1 g per 10 ml of MeOH). The extracts were then filtered, and MeOH was evaporated using a rotary evaporator until only water remained. Sep-Pak C18 cartridges (Waters, Milford, Mass.) were used to fractionate the remaining aqueous extracts. Cartridges were rinsed with 50% H₂O:50% MeOH followed by 20% H₂O:80% MeOH. The 80% MeOH fraction, containing pure flocculosin, is collected and evaporated under a stream of nitrogen or by roto-evaporation. The resulting aqueous phase is lyophilized to obtain the pure compound in powder form.

Identification of Flocculosin

Identification of the glycolipid flocculosin was performed using a 40 mg sample of the pure compound isolated from P. flocculosa. NMR spectra (¹HNMR, HMQC, COSY, TOCSY, ¹³CNMR, DEPT 90, DEPT 135, ³¹P) were recorded on a Bruker WM 600 MHz spectrometer at the Centre Regional de RMN, Département de Chimie, Université de Montréal in methyl alcohol-d4 using TMS as internal standard. ³¹P NMR spectra were recorded in chloroform-d3 using H₃PO₄/D₂O as internal standard, and no signals originating from the glycolipid flocculosin were observed. MS spectra were recorded on a Waters Micro-Mass LCMSMS (75 eV) at the federal research center in Quebec city, and on a FABMS at the centre Régional de Spectrométrie de Masse Département de Chimie Université de Montréal. IR spectra were recorded at Université Laval's Département de Chimie in KBr.

Flocculosin: FABMS: 877.5 (M+Na⁺); LCMSMS 75 eV: 853 (M−, 18), 836 (5), 759 (5), 753 (19), 711 (100), 669 (21), 651 (15), 605 (14), 573 (28), 517 (11), 507 (28), 350 (8), 143 (9); IR: 3422 cm⁻¹, 2926 cm⁻¹, 2854 cm⁻¹, 1744 cm⁻¹, 1246 cm⁻¹, 1073 cm⁻¹, 1044 cm⁻¹. ¹HNMR (MeOH-d4): 5.3-3.3 ppm (19H, m), 2.5 ppm (2H, d), 2.3 ppm (2H, m), 2.2 ppm (3H, s), 2.1 ppm (3H, s), 1.5-1.3 ppm (30H, broad doublet), 1.0 ppm (3H, t); ¹³CNMR (MeOH-d4): 176 ppm, 170 ppm, 170 ppm, 170 ppm, 104 ppm, 101 ppm, 80 ppm, 77 ppm, 75 ppm, 74 ppm, 73 ppm, 73 ppm, 72 ppm, 72 ppm, 70 ppm, 69 ppm, 68 ppm, 68 ppm, 63 ppm, 61 ppm, 43 ppm, 42 ppm, 36 ppm, 32 ppm, 29 ppm (11 superimposed carbon atoms), 25 ppm, 22 ppm, 19 ppm, 19 ppm, 13 ppm

### EXAMPLE II

### Preparation of Flocculosin Derivatives

Protection of Disaccharide Hydroxyl Functions

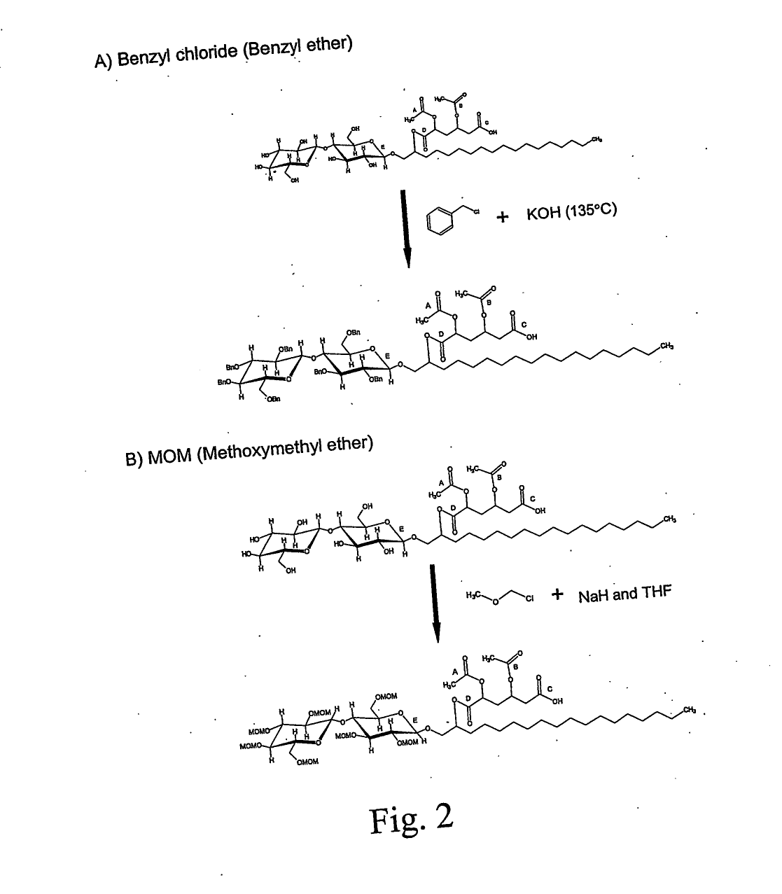

As a first step, the flocculosin is modified by protecting the cellobiose hydroxyl groups (—OH). These functions must be protected before proceeding to other modifications of other functions of the molecule, otherwise the disaccharide moiety will also be affected. Reaction with benzyl chloride (Bn) or methoxymethylester (MOM) under the experimental conditions specified (FIG. 2) enables to achieve O-alkylation on hydroxyl functions of the disaccharide.

This protection reaction is required for the other modifications described below. Deprotection of O-benzyl functions, once derivatives are prepared, is carried out by catalytic hydrogenation in ethanol, whereas the methoxymethylester function is eliminated by using a catalytic amount of hydrochloric acid in methanol.

Alkylation or Acylation of the C Function

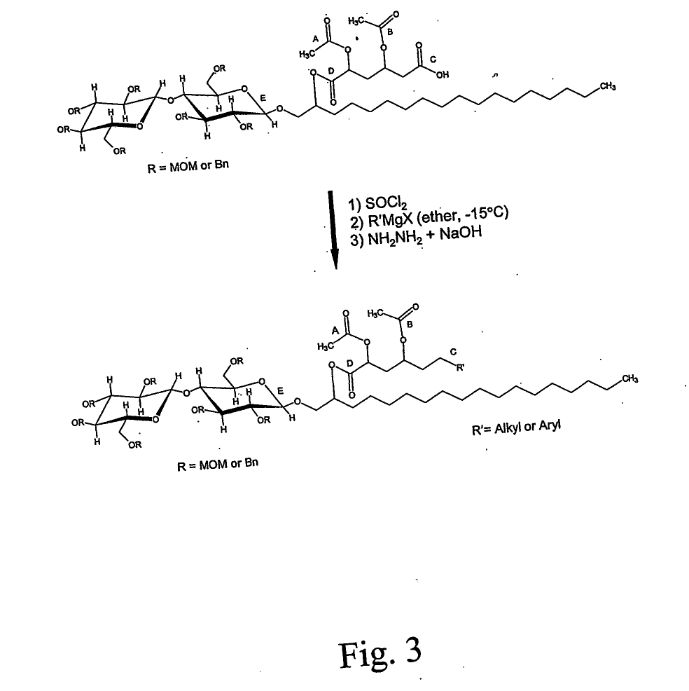

Alkylation or acylation of the C function (FIG. 3) is realized by using thionyl chloride. Reaction of an organomagnesium compound reaction at low temperature on the acid chloride allows the increase in the number of carbon atoms at this position. The new ketone function is then reduced by the Wolf-Kishner reaction (H₂NNH₂ in a basic environment) to completely reduce the ketone function.

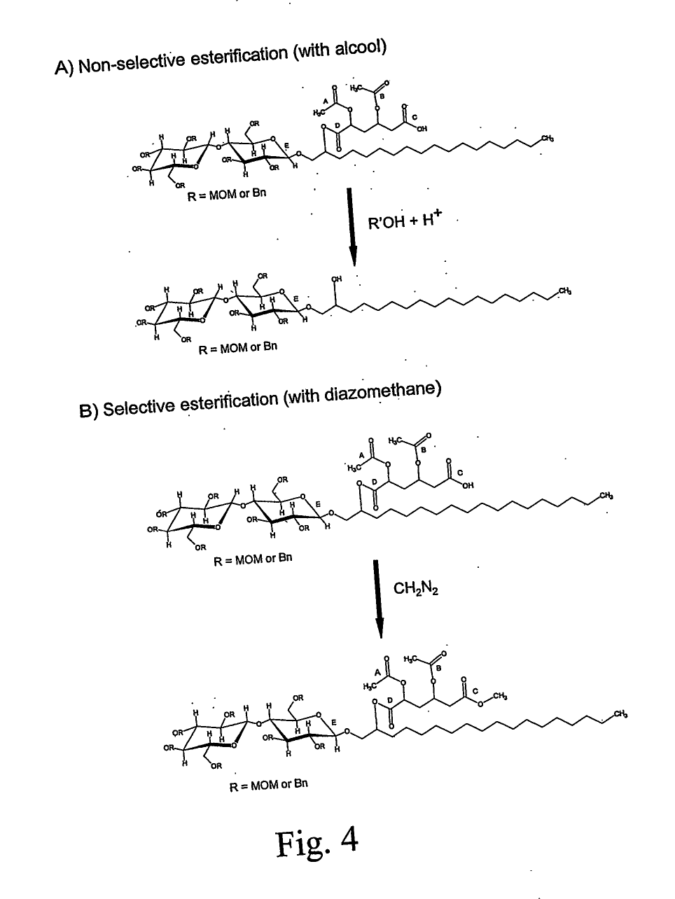

The C function can also be esterified (methylated). A complete (non-selective) esterification will free functions A, B and D leaving the alcohol of position 2 completely free (FIG. 4). A selective esterification (methylation) with diazomethane will yield exclusively the methylester of position 6′. It will be noted that no other function except the methyl function may be added selectively on the acid function.

Amidation/Amination of the C Function

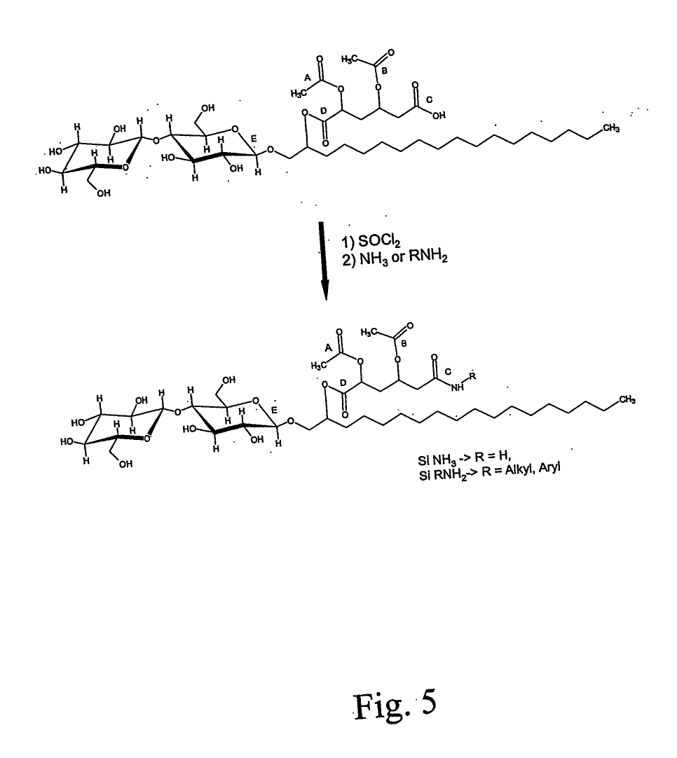

Amidation/amination of the C function can be carried out as follows (FIG. 5). Obtaining an amide in position C is possible, in a selective manner, by previously preparing the acid chloride. The product obtained, which is an amide, could then be reduced into an amine, if desired. It should however be noted that the functions A, B and D can also be handled in the same manner to give the same compound as the one resulting from the esterification described above.

Methylation of Disaccharide Hydroxyl Functions

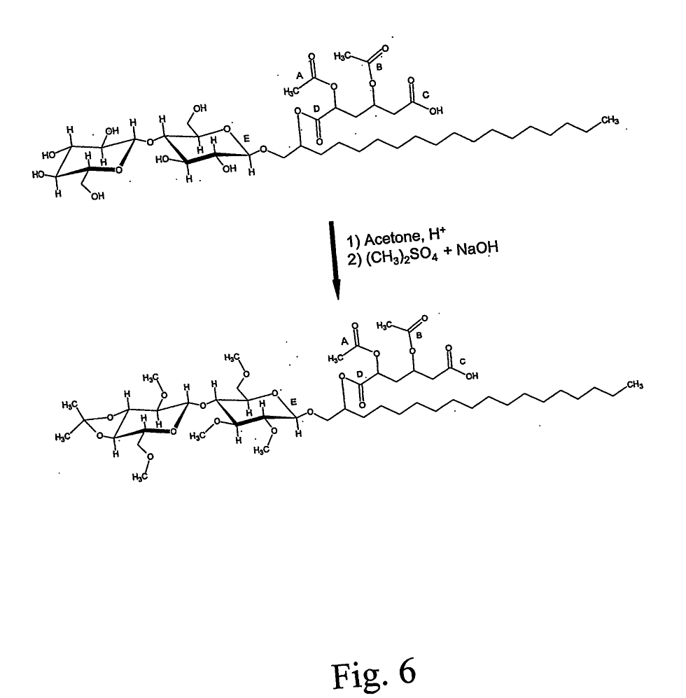

Hydroxyl groups (E) of the cellobiose can be methylated (FIG. 6), by dissolving flocculosin into acetone in a slightly acidic medium, the resulting disaccharide being converted into its acetal derivative (FIG. 6). Selectively, the other functions, including —OH, will be converted into O-methoxy by reaction with dimethylsulfate. The two hydroxyls (as acetal function) could be recovered by light acidification in aqueous phase.

### EXAMPLE III

### Spectrum of Antimicrobial Activity of Flocculosin

To determine the spectrum of activity and potency of the invention, different concentrations (0-1000 μg/ml) of the purified antibiotic were bioassayed with several different strains of infectious microorganisms (see Table 1) using the National Committee for Clinical Laboratory Standard (NCCLS) reference methods for yeasts (M27-A2) or filamentous fungi (M38-A) in 96-well plates. Methanolic solutions containing the different concentrations of flocculosin were added to the medium prior to inoculation with the microorganism selected for bioassay. The inoculated plates were incubated for 3-8 days until inhibition of fungal growth was observed. Minimal inhibitory concentration (MIC) were compiled for each of the tested microorganism (Table 1). MIC is defined as the lowest tested concentration of flocculosin that showed no visible microbial growth.

TABLE 1
Spectrum of antifungal activity of flocculosin

| | Tested organisms | MIC (μg/ml) |
|---|---|---|
| Animal pathogens | Candida albicans | 25 |
| (Including human) | Candida glabatra | 50 |
| | Candida krusei | 50 |
| | Candida lusitaniae | 50 |
| | Candida parapsilosis | 50 |
| | Cryptocuccus neoformans | 50 |
| | Penicillium simpliciossinum | 25 |
| | Saccaromyces cerevisiae | 50 |
| | Trichosporon asahii | 50 |
| Plant pathogens | Aspergillus nidulans | 25 |
| | Botrytis cinerea | 25 |
| | Cladosporium cucumerinum | 25 |
| | Phomopsis sp. | 25 |
| | Phytophthora infestens | 100 |
| | Pythium aphanidermatum | 100 |
| | Pythium sp. | 25 |
| | Rhizoctonia solani | 25 |
| | Slerotinia slerotiorum | 25 |
| | Verticillium lecanii | 25 |

### EXAMPLE IV

### Efficacy of Flocculosin

The efficacy of flocculosin was assessed by time-course testing of Candida albicans (ATCC 18804) with different doses of flocculosin. C. albicans (5×10⁴ cells/ml) was grown in Sabouraud dextrose broth (25° C., 150 rpm). The yeast was treated with 0, 25, 50, and 100 μg/ml of flocculosin. Samples were taken at 0, 30, 60, 90, 120, 180, and 240 min an subjected to plate counts on Sabouraud dextrose agar.

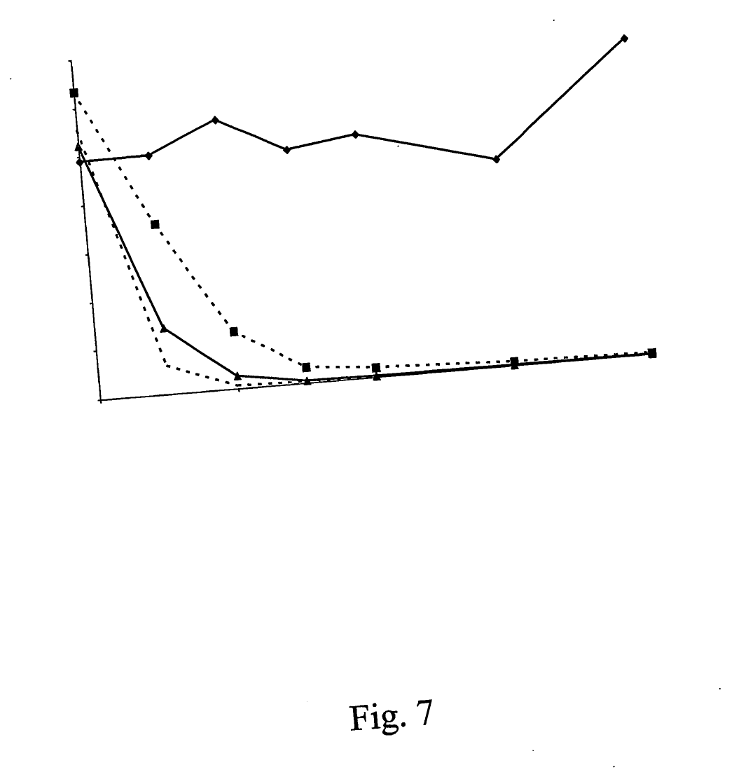

Results demonstrated that the flocculosin treatment were efficient in controlling the growth of C. albicans population when compared to the control (FIG. 7). Increasing the dose of flocculosin caused an overall increase in the speed at which the C. albicans population reached zero (180 min for 25 μg/ml, 90 min for 50 μg/ml, and 60 min for 100 μg/ml of flocculosin).

Overall, these results demonstrate the efficacy of flocculosin as an antimicrobial molecule that acts in a relatively rapid fashion.

### EXAMPLE V

### Association of Flocculosin with Other Antimicrobials

To test the combined effects of flocculosin with other active agents, different doses of flocculosin (0.0005-0.5 μg/ml) was added to different concentrations (0-1 μg/ml) of Amphotericin B in a checkerboard fashion assay and following NCCLS recommendations (NCCLS. Reference Method for Broth Dilution Antifungal Susceptibility Testing of Yeasts. Approved standard-Second Edition. NCCLS document M27-A2, 2002). MIC of Amphotericin B was determined for tested fungi with and without the 5 μg/ml addition of flocculosin and the decrease in MIC of Amphotericin B associated with the addition of flocculosin.

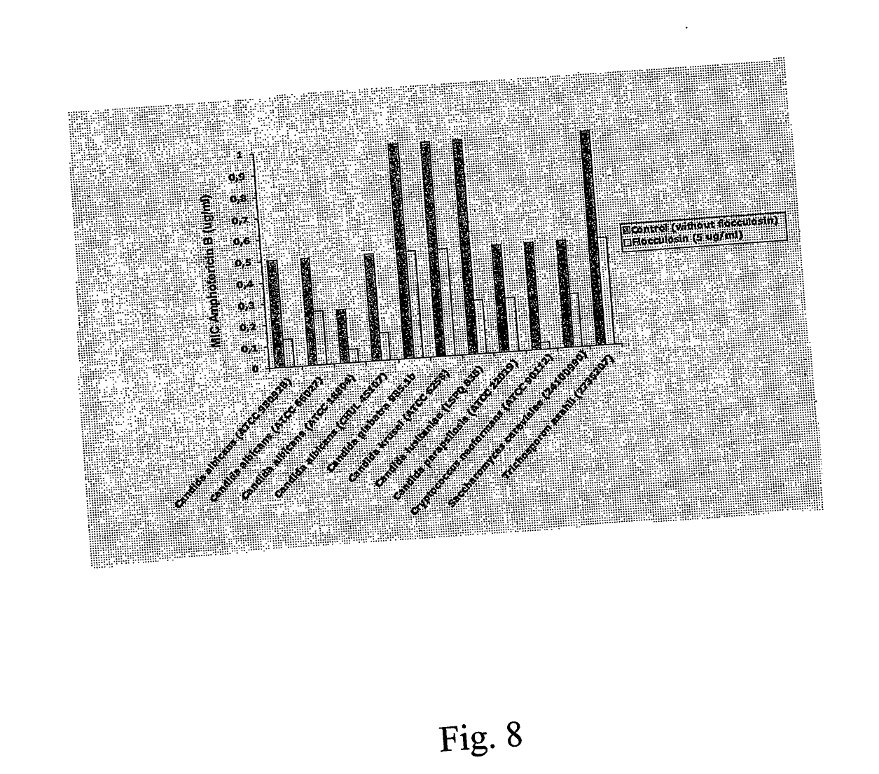

Results demonstrated that the addition of 5 μg/ml of flocculosin significantly decreased the MIC of Amphotericin B in tested microorganisms (FIG. 8). The decrease in Amphotericin B MIC was 50% at the lowest and up to 94% for Candida neoformans.

These results demonstrate the benefic effect of combining flocculosin with other active ingredients.

The invention was exemplified with compounds of formula (2). It has been realized that compounds of formula (3) has the same or even better antimicrobial properties and can be obtained from the flocculosin extracted from P. flocculosa as will be appreciated by one skilled in the art.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be is applied to the essential features hereinbefore set forth, and as follows in the scope of the appended claims.

1. An antimicrobial compound obtained by culturing Pseudozyma flocculosa in a culture medium and characterized by the following NMR and MS spectra: FABMS: 877.5 (M+Na⁺); LCMSMS 75 eV: 853 (M−, 18), 836 (5), 759 (5), 753 (19), 711 (100), 669 (21), 651 (15), 605 (14), 573 (28), 517 (11), 507 (28), 350 (8), 143 (9); IR: 3422 cm⁻¹, 2926 cm⁻¹, 2854 cm⁻¹, 1744 cm⁻¹, 1246 cm⁻¹, 1073 cm⁻¹, 1044 cm⁻¹. ¹HNMR (MeOH-d4): 5.3-3.3 ppm (19H, m), 2.5 ppm (2H, d), 2.3 ppm (2H, m), 2.2 ppm (3H, s), 2.1 ppm (3H, s), 1.5-1.3 ppm (30H, broad doublet), 1.0 ppm (3H, t); ¹³CNMR (MeOH-d4): 176 ppm, 170 ppm, 170 ppm, 170 ppm, 104 ppm, 101 ppm, 80 ppm, 77 ppm, 75 ppm, 74 ppm, 73 ppm, 73 ppm, 72 ppm, 72 ppm, 70 ppm, 69 ppm, 68 ppm, 68 ppm, 63 ppm, 61 ppm, 43 ppm, 42 ppm, 36 ppm, 32 ppm, 29 ppm (11 superimposed carbon atoms), 25 ppm, 22 ppm, 19 ppm, 19 ppm, 13 ppm.

2. An antimicrobial composition comprising an effective antimicrobial amount of the compound or an analog, a derivative or a salt thereof as defined in claim 1.

3. Use of a compound or an analog, a derivative or a salt thereof as defined in claim 1 as an antimicrobial.

4. Use of a compound or an analog, a derivative or a salt thereof as defined in claim 1 in the manufacture of an antimicrobial composition.

5. Use of a compound or an analog, a derivative or a salt thereof as defined in claim 1 for the manufacture of an antimicrobial composition containing said compound or an analog, a derivative or a salt thereof with at least one other active ingredient.

6. A method for the preparation of a compound as defined in claim 1, which comprises the step of cultivating Pseudozyma flocculosa in a culture medium and isolating said compound from the culture medium.
